(19) United States
(12) Patent Application Publication
(10) Pub. No.: US 2024/0285698 A1
(43) Pub. Date: Aug. 29, 2024

(54) METHOD FOR FAVORING WELLNESS AND FACILITATING SELF-CONTROL BEFORE, DURING AND/OR AFTER A RESTRICTIVE DIET

(71) Applicant: DANSTAR FERMENT AG, Zug (CH)

(72) Inventors: Angelo TREMBLAY, Québec (CA); Vicky DRAPEAU, Québec (CA); Thomas Allan TOMPKINS, Québec (CA)

(21) Appl. No.: 18/572,011

(22) PCT Filed: Jun. 23, 2022
(86) PCT No.: PCT/IB2022/055844

§ 371 Date: Dec. 19, 2023

Related U.S. Application Data
(60) Provisional application No. 63/214,103, filed on Jun. 23, 2021.

(51) Int. Cl. A61K 35/747; A23L 33/135; A61P 3/04

(52) U.S. Cl. CPC A61K 35/747; A23L 33/135; A61P 3/04

(57) ABSTRACT

The present disclosure concerns a probiotic composition comprising an effective amount of a probiotic product and an acceptable carrier. The probiotic composition can be used in favoring wellness and facilitating self-control in a subject who had been on a restrictive diet, is currently on the restrictive diet and/or intends to be on the restrictive diet. The probiotic product comprises at least one of a population of probiotic bacteria from a Lactobacillus genus and/or a microbial component derived from the population of the probiotic bacteria.

The present application claims priority from U.S. provisional application 63/214,103 filed on Jun. 23, 2021.

### TECHNOLOGICAL FIELD

The present disclosure relates to a probiotic composition for improving wellness and facilitating control in a subject having received, currently receiving and/or having received a restrictive diet.

### BACKGROUND

Obesity is a worldwide burden with a complex and multifactorial etiology. Over the past few decades, increased consumption of animal-derived fat and highly processed foods and decreased consumption of plant-derived fibers have been pointed out as potential causes for the increasing prevalence of obesity among children and adults.

In addition to directly affecting metabolism, these dietary changes can also significantly impact the gut microbiota composition and function. Beyond the simple correlation between gut dysbiosis, obesity and metabolic diseases, disruptions of host-microbiota interactions in this context were proposed to directly impair the host's metabolism through production of microbial metabolites. However, the bidirectionality of the interaction between dysbiosis and diseases confounds the distinction between cause and consequence.

Biological comorbidities of obesity are the main target of treatments due to their impact on cardiometabolic health and life expectancy, while psychological comorbidities are often neglected even if they can significantly improve health and quality of life. Indeed, there is a link between obesity and mood disorders such as major depression disorder (MDD), stress, anxiety and low self-esteem. Adults with obesity are 55% more likely to develop MDD than non-obese individuals, while depressed adults are 58% more likely to become obese, demonstrating the reciprocity of these disorders. Furthermore, altered eating behaviors such as binge eating, are also common comorbidities of obesity; food addiction is present in 25-37% of individuals with obesity and can reach 60% among individuals with morbid obesity (Luppino et al.).

In other words, the relationship between diet, stress and mood disorders is complex and likely bidirectional, with diet affecting psychological symptoms and psychological symptoms affecting diet with interactions with stress and obesity.

Many hypotheses on the association between obesity and psychological variables have been studied and a focus on the gut-brain axis has emerged in the last few years. The gut communicates with the brain in various ways, including the vagus nerve, microbiota-derived neuroactive compounds, enzymes or gut-derived inflammatory signals. As potential treatments, the concept of psychobiotics, initially defined as “a live organism that, when ingested, confer mental health benefits through interactions with commensal gut bacteria” (Sarkar et al.) has emerged and more studies are now trying to understand the role of the microbiota on the brain.

Sanchez et al. (2017) aimed to demonstrate the impact of synbiotic supplementation (Probiotic+prebiotic, namely Lactobacillus rhamnosus CGMCC1.3724+oligofructose and inulin) on appetite sensations and eating behaviors in the context of a weight-reducing program, but with moderate success.

Although it is accepted in the art that synbiotics are more effective than their probiotic counterparts, such a variety in terms of efficacy on different individuals could be explained by the fact that the prebiotics added to the composition would have stimulated not only the probiotics, but also affected the native host microbiota. In other words, the addition of prebiotics to the Sanchez et al. (2017) formulation could potentially have caused changes in the host microbiota that affected particular host-specific strains, which makes the effect of the synbiotic difficult to predict across subjects (Markowiak et al.).

Thus there remains an acute need for safe, natural and effective strategies and therapies that will prevent, limit and/or suppress the negative psychological effects (alleviation or diminution of food cravings, desire to eat, feeling of hunger, binge-eating, occasional anxiety and stress, etc.) encountered by a subject following a calorie restricted diet.

### BRIEF SUMMARY

The present disclosure provides a probiotic composition (comprising a Lactobacillus sp., a component of a Lactobacillus sp. or a combination thereof for favoring wellness and facilitating self control which may be required in the context of a restrictive diet.

According to a first aspect, the present disclosure concerns a probiotic composition comprising an effective amount of a probiotic product and an acceptable carrier. The probiotic composition is use in favoring wellness and facilitating self-control in a subject who had been on a restrictive diet, is currently on the restrictive diet and/or intends to be on the restrictive diet. The probiotic product comprises at least one of a population of probiotic bacteria from a Lactobacillus genus and/or a microbial component derived from the population of the probiotic bacteria. In an embodiment, the probiotic composition is for preventing, limiting and/or suppressing at least one negative psychological effect associated with the restrictive diet. In another embodiment, the at least one negative psychological effect comprises disinhibition, hunger, a desire to eat, lack of control, intention and planning to consume food, an anticipation of positive reinforcement with food consumption, a binge eating tendency, perceived stress and the anxiety trait, or a combination thereof. In an embodiment, the subject has been determined to need and/or wishes to seek psychological support to complete the restrictive diet. In an embodiment, the subject is an overweight subject. In another embodiment, the subject is an obese subject. In still another embodiment, the subject has a healthy weight. In a further embodiment, the subject is a mammalian subject, such as, for example, a human subject. In still another embodiment, the subject is a male subject. In another embodiment, the subject is a female subject. In an embodiment, the subject is currently on the restrictive diet. In still another embodiment, the population of probiotic bacteria comprises Lactobacillus rhamnosus (L. rhamnosus). In yet another embodiment, the population of probiotic bacteria comprises at least one of L. rhamnosus R0011 (LHS), L. rhamnosus HA-114 (LHS), L. rhamnosus HA-500 (LHS), L. rhamnosus R0049 (LHS), L. rhamnosus R0343 (LHS), or L. rhamnosus R1039 (LHS). In still yet another embodiment, the population of probiotic bacteria comprises L. rhamnosus HA-114 (LHS). In yet another embodiment, the population of the probiotic bacteria is provided at a daily dosage of from about 1×10⁵ to about 1×10¹² colony-forming units (cfu) total bacteria. In still another embodiment, the probiotic composition is provided in the form of a freeze-dried power, a tablet, a capsule, a pill, a suspension, an emulsion, a liquid preparation, a gel, a syrup, a cream or an inhalable formulation. In another embodiment, the probiotic composition is provided as a food product or a drink product.

According to a second aspect, the present disclosure provides a method of favoring wellness and facilitating self-control in a subject in need thereof who had been on a restrictive diet, is currently on the restrictive diet and/or intends to be on the restrictive diet. The method comprises administering to the subject a probiotic composition comprising an effective amount of a probiotic product and an acceptable carrier. The probiotic product comprises at least one of a population of probiotic bacteria from a Lactobacillus genus and/or a microbial component derived from the population of the probiotic bacteria. In an embodiment, the method is for preventing, limiting and/or suppressing at least one negative psychological effect associated with the restrictive diet. In an embodiment, the at least one negative psychological effect comprises disinhibition, hunger, a desire to eat, lack of control, intention and planning to consume food, an anticipation of positive reinforcement with food consumption, a binge eating tendency, perceived stress and the anxiety trait, or a combination thereof. In still a further embodiment, the subject is as defined herein. In a further embodiment, the method comprises administering the probiotic composition during the restrictive diet. In still another embodiment, the probiotic composition and/or the probiotic product is as defined herein.

According to a third aspect, the present disclosure comprises a method of formulating a probiotic composition for favoring wellness and facilitating self-control in a subject in need thereof who had been on a restrictive diet, is currently on the restrictive diet and/or intends to be on the restrictive diet. The method comprises combining (i) at least one of a population of probiotic bacteria from a Lactobacillus genus and/or a microbial component derived from the population of the probiotic bacteria with (ii) an acceptable carrier to prepare the probiotic composition. In an embodiment, the probiotic composition is for preventing, limiting and/or suppressing at least one negative psychological effect associated with the restrictive diet. In yet another embodiment, the at least one negative psychological effect comprises disinhibition, hunger, a desire to eat, lack of control, intention and planning to consume food, an anticipation of positive reinforcement with food consumption, a binge eating tendency, perceived stress and the anxiety trait, or a combination thereof. In a further embodiment, the subject is as defined herein.

According to a fourth aspect, the present disclosure provides the use of a probiotic composition comprising an effective amount of a probiotic product and an acceptable carrier for favoring wellness and facilitating self-control in a subject. The present disclosure also provides the use of a probiotic composition comprising an effective amount of a probiotic product and an acceptable carrier for the manufacture of a medicament favoring wellness and facilitating self-control in a subject. The present disclosure further provides a non-therapeutic use of a probiotic composition comprising an effective amount of a probiotic product and an acceptable carrier for favoring wellness and facilitating self-control in a subject. The subject had been on a restrictive diet, is currently on the restrictive diet and/or intends to be on the restrictive diet. The probiotic product comprises at least one of a population of probiotic bacteria from a Lactobacillus genus and/or a microbial component derived from the population of the probiotic bacteria. In an embodiment, the probiotic composition can be used for preventing, limiting and/or suppressing at least one negative psychological effect associated with the restrictive diet. In an embodiment, at least one negative psychological effect comprises disinhibition, hunger, a desire to eat, lack of control, intention and planning to consume food, an anticipation of positive reinforcement with food consumption, a binge eating tendency, perceived stress and the anxiety trait, or a combination thereof. In an embodiment, the subject is as defined herein. In yet another embodiment, the probiotic composition is for administration during the restrictive diet. In yet another embodiment, the probiotic composition and/or the probiotic product is as defined herein.

### FIGURES

Having thus generally described the nature of the invention, reference will now be made to the accompanying drawings, showing by way of illustration, a preferred embodiment thereof, and in which:

FIG. 1 describes the effect of bacterial strains from Lallemand Health Solutions (LHS) on the decrease of total fat accumulation in Caernorhabditis elegans (C. elegans) wild-type N2. Fat accumulation was measured by fluorescence of Red Nile stained C. elegans wild-type N2 strain fed with 9 LHS bacterial strains or blend, Escherichia coli(E. coli) OP50 (negative control) and E. coli OP50 with orlistat (positive control). ***p-value≤0.001.

FIG. 2 represents the schedule of activities of the clinical trial presented in Example 2.

FIG. 3 depicts the absolute quantification of probiotic (L. rhamnosus HA-114) in fecal samples by quantitative PCR (qPCR) before and after the intervention. n=30 for placebo group and n=37 for probiotic group.

FIG. 4 represents the visual analog scales' results for hunger, fullness, satiety and desire to eat. ITT analyses; n for probiotics at baseline=78, at end of the intervention=69; n for placebo at baseline=72, at end of intervention=63. Data are represented as Mean±SD.

(FIG. 4A) Area under the curve for the standardized breakfast meal.

(FIG. 4B) Area under the curve for the buffet-type lunch meal.

(FIG. 4C) Satiety quotient for the standardized breakfast meal.

(FIG. 4D) Satiety quotient for the buffet-type lunch meal.

FIG. 5 describes the eating and food craving related behaviours' results measured with the Three-Factor Eating Questionnaire, the Binge Eating Scale and the State and Trait Food-Craving Questionnaire mentioned in the STAR methods section. ITT analyses; n for probiotics at baseline=78, at end of the intervention=68; n for placebo at baseline=72, at end of intervention=62. Data are represented as Mean±SD, Within group significance (*=p 50.05, **=p<0.01).

FIG. 6 reports mood scores for body self-esteem, anxiety (state and trait), stress, depression, gastrointestinal symptoms and sleep factors, based on questionnaires mentioned in the STAR methods section. ITT analyses; n for probiotics at baseline=78, at end of the intervention=69; n for placebo at baseline=72, at end of intervention=61. Data are represented as Mean±SD, Within group significance (*=p 50.05, **=p<0.01).

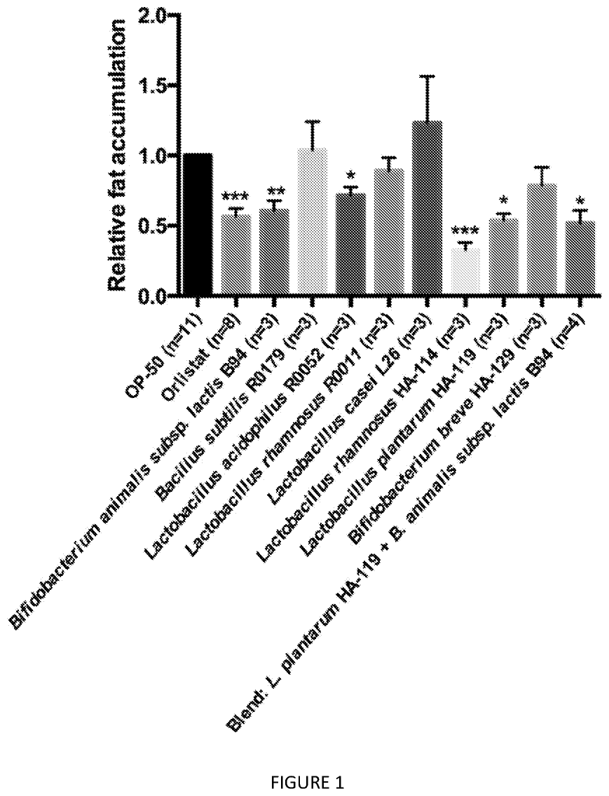

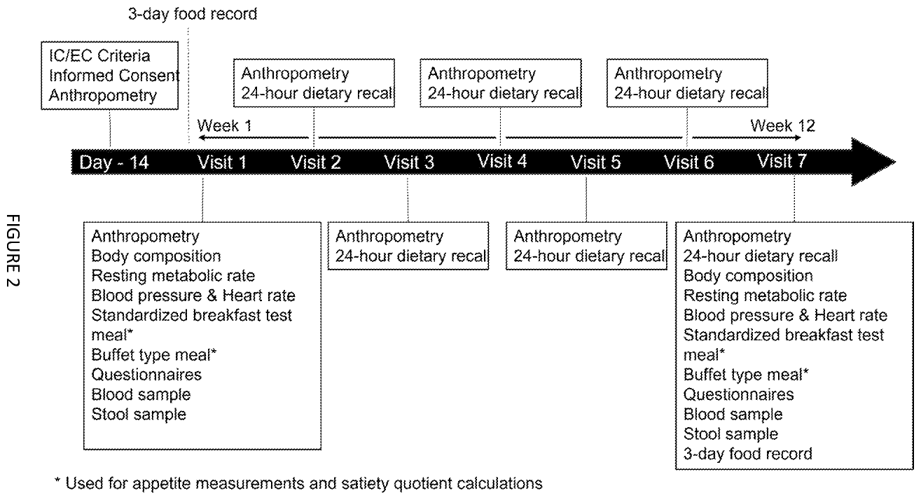

### DETAILED DESCRIPTION

The present disclosure provides the use of a probiotic product comprising and/or being derived from the genus Lactobacillus in improving wellness and facilitating control in a subject having received, currently receiving or having received a restrictive diet. As used in the present disclosure, the expression “improving wellness and facilitating control” is used in reference to a control subject not having received the probiotic product comprising and/or being derived from the genus Lactobacillus. As is apparent from the examples below, it was found that the use of the probiotics Lactobacillus proved to be especially useful for modulating eating and mood-related behaviors associated to a healthier relationship with food and food sensations in a receiving a restrictive diet. More specifically, it was found that the use of the probiotics Lactobacillus proved to be useful for limiting and/or suppressing one or more negative psychological effects which afflict subjects receiving a restrictive diet. It is thus understood that the probiotics Lactobacillus as well as components derived therefrom could be used to achieve similar results in subjects intending to be or having already been on a restrictive diet.

The common negative psychological effects experienced by subjects who having received, currently receiving or having received a restrictive diet comprise, without being limited to, eating behaviours (disinhibition, hunger, binge eating tendencies), food craving related behaviours (desire to eat, lack of control, intention and planning to consume food, anticipation of positive reinforcement with food consumption) as well as mood (anxiety and stress). The one with skills in the art will understand the negative psychological effects listed above as described in Stunkard et al., Hawkins et al., Nijs et al., Beck et al., Spielberger et al. and Cohen et al.. The probiotic composition of the present disclosure can thus be used to prevent, limit and/or suppress at least one (and in some embodiments a combination of) negative psychological effect associated with the restrictive diet. The expression “preventing, limiting and/or suppressing” refer to the ability of the probiotic composition to limit the development, progression and/or symptomology of one or more negative psychological effect associated with the restrictive diet.

The present disclosure concerns subjects who have had, need and/or desire psychological support in order to optimize the success of their restrictive diet. Alternatively or in combination, the probiotic composition can be used with subjects diagnosed with Class 1 obesity (Body Mass Index (BMI) of 30 to <35), Class 2 obesity (BMI of 35 to <40) and Class 3 obesity (BMI of 40 or higher) but also overweight subjects (BMI of 25 to <30). In some further embodiments, the probiotic composition can be used with subjects being in healthy weight range (BMI of 18.5 to <25) who may, in some additional embodiments, have been advised to adjust their diet (e.g., subjects with high cholesterol levels, high blood sugar levels, etc.). In an embodiment, the composition for use of the present disclosure is also dedicated to subjects who do not have metabolic problems but who closely monitor their general health, worrying about the consequences of a possible metabolic problem (e.g., cardiovascular disease, renal disease, etc.) that could appear later on. In another embodiment, the present disclosure is intended for subjects with a healthy weight range who wish to be accompanied in their restrictive diet process, for example to prepare their body for the summer or any other special occasion such as wedding. In a preferred embodiment, the present disclosure aims to make the restrictive diet easier to follow, in other words, probiotic supplementation as described herein makes it possible to ensure/increase compliance with a restrictive diet, whether the subject is followed by a nutrition specialist or not. The subject can be a mammalian subject such as, for example, a human subject. The subject can be a non-human animal. The subject can be a female subject. The subject can be a male subject.

In some embodiments, the subject can use the probiotic composition prior the start of a restrictive diet. As such, the present disclosure provides first administering the probiotic composition to the subject and, afterwards, administering the restrictive diet. Alternatively or in combination, the subject can use the probiotic composition during a restrictive diet.

As such, the present disclosure provides administering the probiotic and the restrictive diet to the subject. Alternatively or in combination, the subject can use the probiotic composition after having completed a restrictive diet. As such, the present disclosure provides administering first the restrictive diet and, afterwards, administering the probiotic composition.

The term “diet” is understood to refer to any kind and amount of food and drink that is ingested by the subject. The terms “restrictive diet” refers as used in the present disclosure to restriction imposed on a subject's diet to limit the kind and/or the amount of food that is prescribed to be ingested. Restrictive diet also includes a restriction on the timing between that food and drink intake. The restrictive diet can be used, in some embodiments, as an attempt to reduce the subject's overall weight and/or fat mass. In some additional embodiments, the restrictive diet can be used to increase the subject's lean mass. In yet additional embodiments, the restrictive diet can be used to modulate the subejct's hormonal balance. The different types of restrictive diets that are accompanied by the supplementation of a composition for use as claimed in the present disclosure can be, for example, low-calorie diets, low-carbohydrate diets (Atkins diet, Dukan diet, etc.), low-fat diets, low-carbohydrate/high-fat diets, high-protein diets, high-carbohydrate/low-fat diets, detox diets (juice fasting, intermittent fasting), liquid diets, diets followed for medical reasons (gluten-free diet, diabetic diet, ketogenic diet, low-fodmap diet, DASH diet (Dietary Approaches to Stop Hypertension), Paleolithic diet, Mediterranean diet, Vegetarian diets, Vegan diets, Flexitarian diets, Food-specific diets (cabbage soup diet, grapefruit diet, etc.), Fasting (intermittent fasting), etc.

Thus, the supplementation of the claimed probiotic composition for use to assist a subject in her/his/its restrictive diet process can be done in a preventive manner in order to prepare for a restrictive diet. Ideally, said supplementation is done in conjunction with a restrictive diet. In another embodiment, the probiotic supplementation is maintained after the restrictive diet in order to minimize or even avoid any risk of relapse. Finally, another embodiment of the present disclosure would be to cumulate the periods of supplementation in a subject wishing to follow a restrictive diet, namely: before, during and after the diet.

The probiotic composition of the present disclosure comprises a probiotic product and an acceptable carrier. In some embodiments, the probiotic product comprises a population of probiotic microorganisms (which may be pure or blended). The term “probiotic microorganism” as used herein refers to a live microorganism which, when administered in adequate amounts, confers a health benefit to the subject having received it. From a regulatory perspective, a probiotic must fulfil several requirements related to lack of toxicity, viability, adhesion and beneficial effects.

In some embodiments, the probiotic product comprises a component derived from the population of probiotic microorganisms. The “component derived from the population of probiotic microorganisms” refers to a biological entity that is produced and optionally secreted by the population of probiotic microorganisms. In some embodiments, the component can be purified/isolated at least in part from the population of probiotic microorganisms. The component can include, without limitation, a nucleic acid residue, an amino acid residue, a carbohydrate, a lipid or a combination thereof. In some embodiments, the component is a fatty acid or a population of different short-chain fatty acids produced and secreted by the population of probiotic microorganisms. In an embodiment, the component can include a medium chain (C6-C12) fatty acid, a long chain (C12-C21) fatty acid, a very long chain (C22 or higher) fatty acid, or a combination thereof. In yet another embodiment, the component can include the one or more of the fatty acids described in Labarre et al., 2020, incorporated herewith in its entirety. Still in the context of the present disclosure, the component when administered in adequate amounts, confers a health benefit to the subject having received it.

In some further embodiments, the probiotic product comprises both the population of probiotic microorganisms and a component derived from the population of probiotic microorganisms.

The probiotic composition can use, as the probiotic microorganism, a probiotic bacteria from the Lactobacillus genus. As used herein, the term “Lactobacillus” refers to members of the genus Lactobacillus, in the family Lactobacillaceae. These bacteria are Gram-positive facultatively anaerobic bacteria that represent a major part of the bacterial group often referred to as “lactic acid bacteria.” Lactobacillus species include, without limitation, L. acidophilus, L. brevis, L. bulgaricus, L. casei, L. crispatus, L. delbrueckii, L. fermentum, L. gasseri, L. helveticus, L. lactis, L. plantarum, L. reuteri, L. rhamnosus, L. salivarius or L. paracasei. While it is not intended that the present disclosure be limited to any particular species of Lactobacillus, exemplary species and strains of Lactobacillus for the present disclosure includes, but are not limited to, the following well-known strains: L. helveticus/L. acidophilus HA-122 (sold by Lallemand Health Solutions (“LHS”)), L. acidophilus R0418 (LHS), L. brevis HA-112 (LHS), L. casei HA-108 (LHS), L. casei R0215 (LHS), L. delbrueckii bulgaricus HA-137 (LHS), L. fermentum HA-179 (LHS), L. helveticus HA-128 (LHS), L. helveticus HA-501 (LHS), L. acidophilus/L. helveticus R0052 (LHS), L. helveticus Lafti L10 R0419 (LHS), L. paracasei HA-196 (LHS), L. paracasei HA-274 (LHS), L. paracasei Lafti L26 R0422 (LHS), L. plantarum R0403 (LHS), L. plantarum R0202 (LHS), L. plantarum R1012 (LHS), L. reuteri HA-188 (LHS), L. rhamnosus HA-114 (LHS), L. rhamnosus HA-500 (LHS), L. rhamnosus R0011 (LHS), L. rhamnosus R0049 (LHS), L. rhamnosus R0343 (LHS), L. rhamnosus R1039 (LHS), L. salivarius HA-118 (LHS), L. salivarius R0078 (LHS), L. bulgaricus R0440 (LHS) or L. lactis R1087 (LHS). In an embodiment, the Lactobacillus is L. rhamnosus. In a further embodiment, L. rhamnosus is L. rhamnosus HA-114 (LHS), L. rhamnosus HA-500 (LHS), L. rhamnosus R0011 (LHS), L. rhamnosus R0049 (LHS), L. rhamnosus R0343 (LHS) or L. rhamnosus R1039 (LHS). In a preferred embodiment, L. rhamnosus is L. rhamnosus HA-114 (LHS) (interchangeably referred to as Lacticaseibacillus rhamnosus HA114 or HA-114). It is intended that the genus include species that have been reclassified (e.g., due to changes in the speciation of organisms as the result of genetic and other investigations) or renamed for marketing and/or other purposes.

In some embodiments, the probiotic composition can include one or more additional probiotic bacteria which are not from the Lactobacillus genus.

The effective amount of colony forming units (“cfu”) for each strain in the composition will be determined by the skilled in the art and will depend upon the final formulation, the subject, etc. The expression “colony forming units” is defined herein as the number of bacterial cells as revealed by microbiological counts on agar plates. For instance, in an embodiment, the total probiotic Lactobacillus provided in a dose of the probiotic composition is at least about 10⁵, 10⁶, 10⁷, 10⁸, 10⁹, 10¹⁰, 10¹¹, 10¹² colony forming units or more. In another embodiment, the total probiotic Lactobacillus provided in a dose of the probiotic composition is no more than about 10¹², 10¹¹, 10¹⁰, 10⁹, 10⁸, 10⁷, 10⁶, 10₅ colony forming units or less. In still a further embodiment, an amount of from about 10⁵ to 10¹² colony forming units (cfu) per daily dose, from about 10⁶ to 10¹² cfu per daily dose, from about 107 to 10¹¹ cfu per daily dose or from about 10⁸ to 10¹¹ cfu per daily dose. In yet another embodiment, the probiotic is provided in an amount greater than about 1.0×10⁹ cfu total probiotic per daily dose. In some embodiments, the subject is administered greater than 3.0×10⁹ cfu total probiotic per daily dose. In another embodiment, the daily dosage can be achieved with one or more administration. However, it is not intended that the present disclosure be limited to a specific dosage as it is contemplated that dosages of total probiotic will vary depending upon a number of factors such as the identity and number of individual probiotic strains employed, the subject being treated and her/his microbiota, the nature of the symptoms suffered by the subject that is to be treated, the general health of the subject, and the form in which the composition is administered.

In one embodiment, the composition for use and methods in accordance with the present disclosure are applicable to humans or more generally to animals. In an embodiment, administration of the composition in accordance with the present disclosure is oral and therefore the present disclosure provides an oral probiotic composition. In another embodiment, administration of the composition in accordance with the present disclosure is parenteral and therefore the present disclosure provides an oral formulation of the probiotic composition. In an embodiment in which the component derived from the population of the probiotic bacteria is intended to be administered, it can be formulated for topical (skin), intranasal, inhalation or sublingual administered and thus the probiotic composition can be formulated as topical (skin), intranasal, inhalation or sublingual formulation.

While it is possible to administer the population of probiotic bacteria of the present disclosure alone, it is typically administered on or in a support (e.g., a carrier) as part of a product, in particular as a component of a food product, a drink product, a dietary supplement, medicament or a pharmaceutical formulation. These products typically contain additional components, acceptable excipients, carriers or adequate additives well known to those skilled in the art. The term “acceptable excipients and carriers” as used herein pertains to those that are compatible with the other ingredients in the formulation and biologically acceptable. In a particular embodiment, the products additionally contain one or more further active agents. In another embodiment, the additional active agent or agents are other probiotic bacteria or yeasts which are not antagonist to the strains forming the composition of the present disclosure. In a particular embodiment, the additional active agent is Cerebiome®, a probiotic formulation developed and provided by LHS and comprising L. helveticus/L. acidophilus R0052 (LHS) and Bifidobacterium longum R0175 (LHS).

Depending on the formulation, the strains may be added as purified bacteria, as a bacterial culture, as part of a bacterial culture, as a bacterial culture which has been post-treated (and at least in part inactivated). In some embodiments, prebiotics, herbal extracts, flavors, vitamins and minerals could be also included in the probiotic composition. However, in some embodiments, it may be beneficial to provide a probiotic composition lacking prebiotics. As such, in some embodiments, the probiotic composition of the present disclosure does not include (e.g., it excludes) prebiotics (it can be considered “prebiotic free”). However, in alternative embodiments, the probiotic composition can include one or more prebiotic to maintain the viability of the population of probiotic bacteria during storage. In other embodiments, the probiotic composition can be provided as an additive to a food product, a dietary supplement or a medicine or pharmaceutical formulation. Care must be taken during the formulation of the probiotic composition not to negatively affect the bioavailability of the probiotic product forming the composition and is within the scope of ordinary persons skilled in the art. In some additional embodiments in which a viable population of probiotic bacteria is provided as the probiotic product, care should also be taken not to substantially affect the bioavailability of the probiotic product.

For example, the probiotic composition of the present disclosure can be formulated in the form of freeze-dried power, tablet, capsules, pills, suspension, lozenge, emulsion, liquid preparations, gel, syrup, cream, ointment, inhalable product, etc.

The probiotic composition of the present disclosure can be used as an ingredient in food products such as milk products, yogurt, curd, cheese (e.g., quark, cream, processed, soft and hard), fermented milk, milk powder, milk based fermented product, ice-cream, a fermented cereal based product, milk-based powder (such as for example a whey concentrate), a beverage, a dressing, meat products (e.g. liver paste, frankfurter and salami sausages or meat spreads), spreads, fillings, frostings, chocolate, confectionery (e.g. caramel, candy, fondants or toffee), baked goods (cakes, pastries), sauces and soups, fruit juices or coffee whiteners.

The present disclosure further provides a method for making the probiotic composition described herein. The method first provides cultivating the probiotic microorganisms in a suitable medium and under suitable conditions as known in the art. The probiotic microorganisms can be cultivated alone to form a pure culture, or as a mixed culture together with other microorganisms, or by cultivating probiotic microorganisms of different types separately and then combining them in the desired proportions. After cultivation, and optionally until a predetermined CFU/g concentration is reached, a cell suspension is recovered and used as such or treated in the desired manner, for instance, by concentrating, spray drying, lyophilization, flatbed oven drying, freezing or freeze-drying, to be further employed in the preparation of composition and can be blend with a carrier medium. Viability assays can be performed at different steps of the production method (i.e., after cells propagation, after formulation of the probiotics and/or after storage periods of the final product) to ensure good quality control of the product using common techniques known in the art, such as, for example, flow cytometry. In some embodiments, the method can include purifying, at least in part, the component from the propagated population of probiotic bacteria. Sometimes the probiotic preparation is subjected to an immobilization or encapsulation process in order to improve its shelf life and/or gastro-resistant and/or extended release properties. Several techniques for immobilization or encapsulation of bacteria are known in the art. Once the population of probiotic bacteria and/or the component has been obtained it can be admixed with an acceptable carrier and formulated as a solid dosage form, a liquid dosage form or a gaseous dosage form. In some embodiments, the population of probiotic bacteria and/or the component is formulated as a food product, a drink product or a feed product. Once formulated, the probiotic can be stored prior to being administered to the subject.

In the probiotic composition of the present disclosure, the population of probiotic bacteria can be provided in the form of viable cells or in an inactivated form of non-viable cells (i.e., killed cultures). As such, in some embodiments, the method can include submitted the population of probiotic bacteria to a thermal (heat or cold) treatment, a pH treatment, a radiation (sonication) treatment and/or a pressure treatment to kill the viable cells of the population of probiotic bacteria.

In accordance with the present disclosure, probiotic compositions are intended be administered so that a symptom-ameliorating effective daily dose is received by the subject. The daily dose may be given in divided doses as necessary, the precise amount of the compound or agent received and the route of administration depending on the general health of the subject being treated according to principles known in the art. A typical dosage regime is once, twice or thrice daily. In some embodiments, the probiotic compositions can be administered at least two consecutive days. In some further embodiments, the probiotic composition can be administered at least two, three, four, five, six or seven consecutive days in a defined period. The defined period of administration can be repeated more than once until the desired end-point is achieved. The probiotic composition can be further administered as one or more further (maintenance dose). In some embodiments, the probiotic composition can be administered at least once a week or at least once a month to provide the one or more maintenance dose.

The word “comprising” in the claims may be replaced by “consisting essentially of” or with “consisting of,” according to standard practice in patent law.

The present invention will be more readily understood by referring to the following examples which are given to illustrate the invention rather than to limit its scope.

### EXAMPLES

### Example 1: Screening of Probiotic Strains for Fat-Accumulation Activity in Caenorhabditis elegans

There is a reciprocal relation between obesity and mood disorders, the presence of one increases the likelihood of observing the other in the same patient. On the one hand, cross-sectional studies have shown that patients with mood disorders, such as depression or a manic episode, are more likely to become obese compared to the general population. On the other hand, patients suffering with obesity are more likely to report an episode of major depressive disorder, showing the reciprocity between the two disorders (Soczynska et al.).

An important link between mood disorders and obesity are the high circulating levels of proinflammatory cytokines, such as TNF-α, IL-1β and IL-6. Indeed, patients suffering with obesity and/or mood disorders show aberrant microglial activation which results in the release of inflammatory cytokines, such as TNF-α and BDNF. Furthermore, animal studies have shown that proinflammatory cytokines such as TNF-α and IL-6 can be synthesized by the adipose tissue, while other hormones released by the adipose tissue stimulate systemic proinflammatory release (Soczynska et al.). Therefore, fat accumulation deeply modulates systemic inflammation among participants with mood disorders and/or obesity.

Considering that fat accumulation modules not only obesity, but also mood disorders, a number of probiotic strains provided by Lallemand Health Solutions (LHS) were evaluated for whether or not they can affect the storage of fat in the model organism N2 C. elegans. The study protocol below was inspired from the fat accumulation protocol proposed in Grompone et al.

### Material and Method

The probiotic strains considered in this comparative study are Lactobacillus rhamnosus HA-114, Lactobacillus plantarum HA-119, Lactobacillus paracasei HA-196, Bifidobacterium breve HA-129, Lactobacillus plantarum R1012, Bifidobacterium animalis subsp. lactis B94 (R0421), Bacillus subtilis R0179, Lactobacillus acidophilus/helveticus R0052, Lactobacillus rhamnosus R0011, Lactobacillus casei L26 and a blend consisting of L. plantarum HA-119+B. animalis subsp. lactis B94 (R0421). Orlistat and E. coli OP50 were chosen as positive and negative control, respectively.

Wild type N2 C. elegans worms were first age-synchronized by letting them grow on 4×10 plates until plenty of eggs/gravid adults were observed. Worms were then collected with 1.5 mL M9 buffer and centrifuged (4 min, 3400 g). Supernatants were discarded and pellets were resuspended in 15 mL M9 buffer. Centrifugation step was repeated once, and 3 mL supernatant was replaced with 3 mL 2:1 bleach:NaOH 1M solution. Entire suspension was vortexed for 5-10 min prior to centrifugation, and 9 mL supernatant was replaced with fresh 9 mL M9 buffer. This step was repeated 3 times. After the last centrifugation step, supernatants were discarded, and the pellets were resuspended in 250 μL M9 buffer. Worm suspensions were deposited on a plate without any bacteria but with Nile red (0.20 μg/mL) or Nile red (0.20 μg/mL)+Orlistat (12 μg/mL) and were allowed to dry overnight at 20° C.

In a second part of the experiment, worms were grown with probiotics. Worms were collected at L1 stage with 1 mL M9 buffer per plate and split on different plates containing either probiotic bacteria or E. coli OP50. The plates were then stored away from light for 2-3 days, allowing the worms to reach adulthood. About 200 adult worms from each condition tested were collected in Eppendorf tubes with 750 μL M9 buffer. Eppendorf tubes were centrifuged at 11000 g for 4 min, supernatant was discarded leaving around 100 μL M9 buffer and 100 μL of these suspensions were transferred in a black 96-well plate. Fat accumulation into worms (fluorescence) was measured for each condition using a plate reader (480 nm/571 nm, sensitivity=150) whereas remaining worms (from plates and Eppendorf tubes) were counted.

### Results

Nile red fluorescence analysis of N2 worms fed with E. coli OP50 with Orlistat showed a significant decrease of fat accumulation, confirming that Orlistat is an effective positive control. Worms fed with Bacillus subtilis R0179, Lactobacillus rhamnosus R0011, Lactobacillus casei L26 and Bifidobacterium breve HA-129 showed similar levels of Nile red fluorescence as worms fed with E. coli OP50. Interestingly, worms fed with, Lactobacillus acidophilus/helveticus R0052, Lactobacillus plantarum HA-119, Blend: Lactobacillus plantarum HA-119+Bifidobacterium animalis subsp.lactis B94, Bifidobacterium animalis subsp. lactis B94 and more particularly Lactobacillus rhamnosus HA-114 showed a significant or a highly significant decrease of fat accumulation vs. negative control. Interestingly, it was also noticed that the 2 strains of L. rhamnosus used in these experiments gave very opposite results. Thus, based on known characteristics of this genera and on preliminary data showing L. rhamnosus HA-114 lowered body weight gain and fat accumulation more efficiently than other strains in C. elegans, it has been decided to investigate this probiotic's potential to decrease weight in combination with a diet among otherwise healthy adult participants with overweight or obesity. In addition, based on the commonalties and the high comorbidity between the obesity and mood disorders, it was decided to evaluate the impact of the probiotic and nutritional intervention in mood disorders and eating behaviors among patients with obesity and overweight.

### Example 2: Clinical Trial

This study, a randomized, double-blind, placebo-controlled, two-arm, parallel groups clinical trial assessed biological markers of energy metabolism and behavioral/psychological variables (such as eating-behaviors, appetite sensations and mood) in 152 overweight/obese adults receiving L. rhamnosus HA-114 supplementation combined with a 12-week dietary intervention inducing a controlled weight loss.

### Participants

Subjects were stratified based on gender and Body Mass Index (BMI), then randomly assigned to receive either probiotic (L. rhamnosus HA-114) capsules or an appearance- and taste-matched placebo capsule provided by Lallemand Health Solutions (LHS; Montreal, Quebec, Canada). Participants were recruited between January 2017 and March 2019 in Quebec City, through advertisement on campus.

To meet the inclusion criteria participants needed to be aged between 18 and 55 years old, have a BMI between 27.0 and 39.9 kg/m², be sedentary to moderately active (less than 30 minutes of physical activity, 3 times per week), willing and able to provide informed consent as well as committed to losing weight over the 12-week study period. Willingness to complete questionnaires, records, and diaries associated with the study, to complete all study visits, discontinue consumption of fermented foods or, laxatives, prebiotics and any substance for body weight control, receive random assignment to probiotic or placebo supplementation was also taken into account.

Exclusion criteria included the use of another investigational product or having had a weight gain or loss of at least 10 lbs within three months of the pre-baseline period, participating in another weight loss program or taking a medication for weight loss at enrollment or at any point during the study, antibiotics intake or being under any treatments (medication or nutritional program) affecting body weight, food intake and/or energy expenditure, smoking or a history of drug and/or alcohol abuse (>9 drinks weekly). Women recruited could not be in menopause, or known to be pregnant, breastfeeding, or planning on becoming pregnant in the next 18 months.

Any of the following health conditions also resulted in exclusion: inflammatory bowel syndrome, celiac disease, short bowel syndrome or any other malabsorptive syndrome, uncontrolled angina within the past six months, insulin-dependent diabetes (oral medications are not exclusionary), serious and/or unstable medical conditions (e.g. cardiovascular, renal, lung, psychiatric illness, bleeding disorders, etc.), cancer treatment (radiation, chemotherapy, surgery) within past six months or any other treatment or condition known to weaken the immune system, any physical condition deemed likely to interfere with individuals' ability to participate in a nutritional intervention, allergy to milk, soy, or yeast, abnormal thyroid hormone levels, immuno-compromised conditions or participant experiencing nausea, fever, vomiting, bloody diarrhea or severe abdominal pain.

### Intervention

The probiotic, L. rhamnosus HA-114, was given under the form of freeze-dried bacteria capsules (˜10⁸ to ˜10¹⁰ CFU per capsule), provided by Lallemand Health Solutions (LHS; Montreal, Quebec, Canada) and stored refrigerated (2-8° C.). Excipients (non-medicinal and inert ingredients) found in the probiotics and placebo capsules were potato starch, hydroxypropylmethylcellulose (HPMC), titanium dioxide, and magnesium stearate. Participants were instructed to take the capsule daily, with a meal at the same time. In the event that a participant forgot to take a capsule, he or she was instructed to take the missed dose as soon as they remembered but were not to exceed more than one capsule per day. Participants were instructed to keep the product refrigerated during the study.

Dietary intervention consisted in a 12-week food plan, inducing a daily caloric restriction of 500 kcal, during the same period they consumed the probiotic formulation or the placebo. The energy content of the diet prescription was customized to each participant (resting metabolic rate (RMR)×physical activity level (PAL)−500 kcal). In order to follow the prescription, participants were given a diet-exchange nutrition plan which met the calculated energy intake±20 kcal also includes targets in the macronutrient composition (45-50% carbohydrates, 25-30% lipids, 20-25% proteins) in order to promote a healthy and satiating diet. It should also be mentioned that nutritionists, as all professionals carrying out a clinical trial, must abide by ethical guidelines of beneficence and non-maleficence by providing proper nutritional guidance, and preventing malnutrition. To create a supportive and motivating environment and to whether assess dietary recommendations were still adequate and respected, participants came for follow-up visits every two weeks.

Fecal L. rhamnosus HA-114 Detection

Stool samples were obtained before and after the 3-month protocol from a subgroup of participants (placebo, n=30 and probiotic, n=37), aliquoted in sterile tubes and frozen within 4 hours of collection. DNA was extracted from 150-250 mg of homogenized stool samples using the ZymoBIOMICS 96 MagBead DNA kit (Zymo Research, cat #D4308), automated on the KingFisher Flex Purification System with the 96 Deep-well head (ThermoFisher). The bead beating step consisted of five 1-minute rounds of bead beating, with 1-minute intervals of rest in between each using the FastPrep-24 5G bead beating grinder and lysis system (MP Bio). Modifications to the manufacturer's protocol included: 1) The addition of 0.5% v/v of β-mercaptoethanol to the MagBinding Buffer and 2) the use of 900 μL ZymoBIOMICS™ MagWash 1. The automated settings for each step can be found in a.bdz format using the following link: https://github.com/Zymo-Research/KingFisher.Flex.QuickDNAFecal. DNA purity and yield were assessed using a NanoDrop One UV/Vis spectrophotometer (ThermoFisher Scientific).

The absolute quantification of L. rhamnosus HA-114 was achieved using the CFX384 Touch Real-Time PCR Detection System (Bio-Rad Laboratories) according to previously described methods (Ford et al.) using L. rhamnosus HA-114 specific primers.

### Physiological Parameters' Measurements

At baseline and at the end of the intervention (12 weeks±1 week), participants came for 5 h visits starting in the morning after a minimum of 12 h fasting, 24 h without intense physical activity and 48 h without alcohol consumption. During these visits, physiological parameters such as blood and anthropometric parameters, blood pressure, heart rate, resting metabolic rate were measured, as well as appetite sensations before and after a standardized breakfast test meal and a lunch buffet-type meal. Participants also completed questionnaires assessing psychological parameters such as eating behaviors, food craving related behaviors and mood (FIG. 2).

### Resting Metabolic Rate (RMR)

RMR was measured by indirect calorimetry after a 12-hour overnight fast. After a 15-min resting period, indirect calorimetry measurements were performed using an open circuit system over a 15-min period. Gases were collected, analyzed and quantified using the Weir formula to determine the energy equivalent of 02 volume. The determination of substrate oxidation was assessed through the calculations previously described by Frayn et al. while assuming that protein oxidation contributes to 10% of total energy expenditure measured under these conditions.

### Anthropometric Parameter, Body Composition Measurements, Blood Pressure and Heart Rate

Body weight was performed with a scale (Body Composition Monitor, Tanita), without shoes and with light clothing. Height was measured using a Stadiometer Holtain in a standing position, considering the total distance between participants' top of their head and the ground on which (s)he stands to a 1-mm precision. Waist circumference was measured at the level of the minimum size of the abdomen, located halfway between the bottom of the ribs and the iliac crest, while the participant was standing. This measurement was performed using a fiberglass tape measure (Gulick) graduated in centimeters and displayed with 1-mm precision. Body composition was assessed by dual energy absorptiometry with X-rays (DXA; Prodigy Bone Densitometer System, GE Lunar Corporation). Blood pressure and heart rate were measured in a fasted state, on the right arm after a 5-min resting period with an automatic blood pressure monitor (Physio Logic Essentia, 106-930 by AMG Medical Inc.).

### Standardized Breakfast Test Meal

Subjects were asked to come to the laboratory after a 12-hour overnight fast for a standardized breakfast test meal. The standardized breakfast consisted of white bread, butter, peanut butter, cheddar cheese, and orange juice. The meal was designed to have a food quotient of 0.85 and an energy content of 733 kcal for men and 599 kcal for women. Subjects were instructed to eat each meal within a 20-minute period. Appetite sensations measurement were performed before and after the standardized breakfast.

### Buffet-Type Meal

Participants were asked to return 3 h30 minutes from the end of breakfast to eat a buffet-type meal composed of 32 different food items in order to assess energy intake and macronutrient preferences. Subjects had 30 minutes to eat lunch. All food items were served ad libitum and were weighted before and after the subject consumption. The Canadian Nutrient File (2015) was used to evaluate total energy intake and macronutrient composition at the lunch meal.

### Psychological Parameters' Measurements

To successfully lose weight, a dieter requires strong self-regulation which is intrinsically associated to executive functions (Dohle, et al.). Executive function is an umbrella term that encompasses higher-order, goal-oriented cognitive processes, such as working memory representations, impulse inhibition and shifting attention from one task to another (Dohle, et al.). Dieters require an efficient representation of their long-term goal (i.e., weight loss) in order to redirect their attention away from a high-fat, desired meal and not follow their impulse of consuming it (Dohle et al.). Redirecting attention is a method to ease control of inhibition, a self-regulatory behavior that prevents dieters from acting impulsively (Dohle, et al.). While inhibitory control is not associated to an increased consumption of healthier meals, it certainly is associated to decreased likelihood of unhealthy foods intake (Dohle et al.). Another important executive function is task-switching as it allows dieters to assess dieting methods and adapt them to achieve the goals more efficiently, facilitating self-regulation.

### Appetite Sensation Measurements and Satiety Quotient (SQ) Calculation

Desire to eat, hunger, fullness and prospective food consumption (PFC) were rated immediately before (after a 12 h overnight fast) and after (0, 10, 20, 30, 40, 50, 60 and 120 minutes) the standardized breakfast test meal as well as before and after (0, 60, 120, 180, 240 minutes) the buffet-type meal on a 150-mm visual analogue scale (VAS) adapted from Hill and Blundell. Questions were asked as follows: 1) How strong is your desire to eat? (Very weak-Very strong); 2) How hungry do you feel? (Not hungry at all-As hungry as I have ever felt); 3) How full do you feel? (Not full at all-Very full), and 4) How much food do you think you could eat? (Nothing at all-A large amount). VAS measurements were always performed in the same environment, i.e. at the same table with the same lighting in the same room which was kept free of odors and sounds as well as other potentially confounding factors (visual stimuli, individuals in the room, etc.). The satiety capacity of each individual referred to as the SQ for each appetite sensation were calculated with this equation adapted from Green et al.: SQ=(fasting appetite sensations—mean of the post meal appetite sensations)/caloric content of the meal×100. Thus, the SQ is expressed in mm/kcal. Unlike the SQ equation proposed by Green et al., we used the mean 60 minutes post-meal appetite sensations to represent a more stable post-meal response to the caloric load (Drapeau et al., 2005 & 2007). In order to investigate the SQ over time, a VAS measurement for appetite sensation was added two hours after the breakfast.

### Eating Behaviors Questionnaires

Eating behaviors were measured by the Three Factor Eating Questionnaire (TFEQ; Stunkard et al.) that measures three main dimensions of human eating behaviors, cognitive restraint, disinhibition and susceptibility for hunger, the Binge Eating Scale (Hawkins et al.) which assesses eating-related working memory, inhibition control and task-switch related to binge eating tendencies as well as the Food Cravings Questionnaire (FCQ; Nijs et al.) which differentiates State (S) and Trait (T) through different parameters such as lack of control, desire to eat, intention and planning to consume food, the anticipation of positive reinforcement with food consumption and relief from negative state.

### Mood-Related Questionnaires

Wellness and quality of life was assessed with the following questionnaires: the Body Esteem Scale (Mendelson et al.) to measure distress-related body esteem, the Beck Depression Inventory (Beck et al.) which evaluates depression symptoms, the State-Trait Anxiety Inventory (Spielberger et al.), to measure anxiety symptoms, the Perceived Stress Scale (Cohen et al.) to evaluate the level of stress, the Pittsburgh Sleep Quality Index (Buysse et al.) to assess sleeping quality and the Gastrointestinal Symptom Rating Scale (GSRS; Svedlund et al.) to assess gastrointestinal symptoms including diarrhea, constipation, acid reflux, indigestion, and abdominal pain.

### Biochemical Analysis

Blood samples were drawn after a 12-hour overnight fast from an antecubital vein through a venous catheter. The following blood parameters were measured in the fasted state: glucose, insulin, total cholesterol, HDL-cholesterol, LDL-cholesterol, triglycerides, ALT and AST, C-reactive protein, estradiol, leptin and total ghrelin. Glucose, total cholesterol, HDL-cholesterol, LDL-cholesterol, ALT and AST determinations were performed by a portable chemistry analyzer (Piccolo Xpress, Abaxis inc.); C-reactive protein, insulin and estradiol measurements have been performed at the biochemical analysis platform of the Quebec Heart and Lung Institute; ghrelin and leptin were assessed by ELISA according to manufacturer's instructions (Human Leptin Instant ELISA Kit and Human Ghrelin ELISA Kit, Invitrogen, ThermoFisher Scientific).

### Statistics

The treatment effect was estimated using a linear mixed ANCOVA model adjusting for the effect of relevant covariates, gender and age, in the model. Appropriate non-parametric methods were applied if the data significantly deviated from the normality assumptions. For each endpoint, a single statistical model based on the experimental design and which includes effects for time and treatment were constructed. The fixed effects in the statistical model are time point (visit), treatment group (probiotic or placebo) and their interaction. No imputation was performed for missing observations. Statistical models that account for unbalanced data were used instead. The analyses were conducted on the intention-to-treat population using JMP (v14.1.0). Statistical significance was set at 5% and no corrections of significance level was applied to adjust for multiple testing.

Power analysis was performed based on weight loss, using a t-test (assuming approximate normality) at a significance level of 5%, statistical power of 85%, and estimated a standard deviation of 2.7 kg for both groups. The power calculations for this two-arm study suggested that 120 participants (60 per group) were required to reach statistical significance of the primary aim. In addition, a dropout rate of approx. 25% was anticipated. Thus, a total number of 152 overweight/obese participants were recruited into the study.

### Results

Fecal L. rhamnosus HA-114 Detection

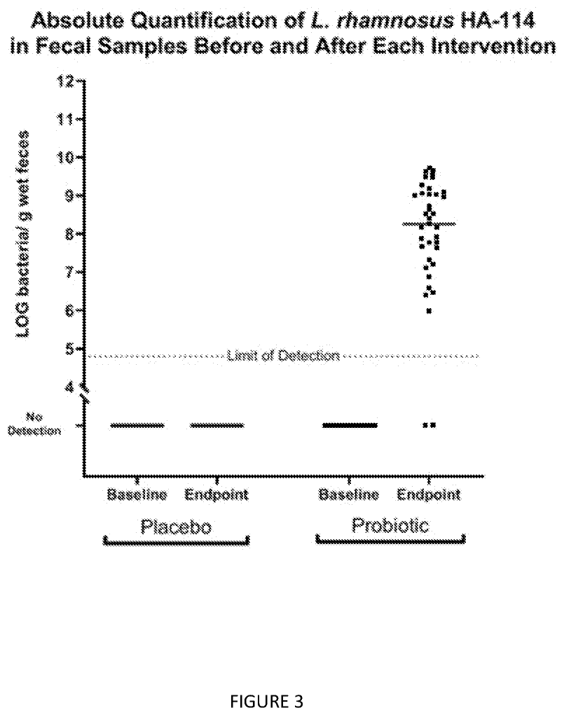

L. rhamnosus HA-114 was not detected in baseline stool samples using strain-specific qPCR assays. At the end of the intervention, the strain was not detectable in any of the fecal samples provided by participants in the placebo group, while it was recovered and quantified in 35 of the 37 samples from the probiotic group (FIG. 3).

### Body Weight and Body Composition

The dietary intervention, aiming at a decrease of 500 kcal/day in energy intake, reduced body weight, body mass index, waist circumference, fat mass and percent body fat, whereas no significant modification in lean mass was induced by the protocol (Table 1). Although on average the placebo group lost 2.96 kg of fat mass compared to 3.24 kg for the probiotic group, there was no significant interaction with the probiotic supplementation (p>0.05).

### Circulating Markers, Blood Pressure and Heart Rate

Multiple blood markers related to metabolic and general health were measured in fasted blood samples collected at baseline and at the end of the intervention (Table 1). As participants were healthy and did not suffer from severe illnesses, including insulin-dependent diabetes, circulating levels of markers such as plasma triglycerides, cholesterol and c-reactive protein did not reach clinical diagnostics levels. In a similar way, blood pressure and resting heart rate were considered normal. Interestingly, plasma insulin, LDL-cholesterol and triglycerides levels were decreased over time in the probiotic group only, although there was no significant interaction between diet and treatment. Leptin and ghrelin, which are hunger-related hormones produced by the adipose tissue and gut, respectively, were measured in the fasted state. Leptin decreased in both groups while ghrelin significantly increased in the probiotic group only.

TABLE 1
Anthropometric measurements, body composition changes, fasting blood markers, pressure and heart
rate. Data are represented as Mean ± SD, Within group significance (*= p < 0.05, **= p < 0.01).

| | Placebo | | Probiotic | |
|---|---|---|---|---|
| | Baseline | 3 months | Baseline | 3 months |
| | (n = 72) | (n = 50) | (n = 78) | (n = 54) |
| Body weight (kg) | 89.0 ± 13.3 | 84.8 ± 12.3** | 89.2 ± 12.1 | 84.2 ± 10.2** |
| Body weight loss (kg) | 88.9 ± 13.3 | 84.8 ± 12.3** | 89.2 ± 12.1 | 84.2 ± 10.2** |
| Body Mass Index (kg/m²) | 31.7 ± 2.8 | 30.3 ± 2.7** | 32.2 ± 3.1 | 30.7 ± 3.2** |
| Waist circumference (cm) | 96.6 ± 9.8 | 93.7 ± 9.3** | 96.3 ± 8.9 | 92.5 ± 8.3** |
| Fat mass (kg) | 35.9 ± 7.3 | 32.7 ± 7.0** | 37.0 ± 7.1 | 33.5 ± 7.7** |
| Lean mass (kg) | 48.8 ± 9.5 | 48.4 ± 9.7 | 47.8 ± 9.6 | 47.2 ± 9.0 |
| Body fat (%) | 41.1 ± 6.4 | 39.1 ± 6.9** | 42.4 ± 6.7 | 40.1 ± 7.7** |
| Insulin (pmol/L) | 76.1 ± 37.9 | 73.2 ± 44.9 | 67.1 ± 38.8 | 58.7 ± 35.6** |
| Glucose (mgl/dL) | 96.6 ± 13.7 | 96.9 ± 8.1 | 94.0 ± 7.5 | 93.8 ± 6.0 |
| LDL-Cholesterol (mg/dL) | 131.4 ± 35.6 | 128.9 ± 32.8 | 124.0 ± 34.0 | 118.2 ± 34.4* |
| HDL-Cholesterol (mg/dL) | 53.9 ± 10.6 | 51.5 ± 10.4 | 57.3 ± 13.8 | 53.7 ± 12.6 |
| Total Cholesterol (mg/dL) | 182.8 ± 38.6 | 180.4 ± 32.5 | 182.3 ± 38.1 | 172.1 ± 35.9 |
| Triglycerides (mg/dL) | 122.0 ± 57.5 | 115.1 ± 64.6 | 107.6 ± 55.1 | 95.9 ± 44.4** |
| Leptin (ng/mL) | 14.6 ± 9.5 | 10.5 ± 9.0** | 15.1 ± 10.5 | 11.1 ± 9.2* |
| Total Ghrelin (ng/mL) | 4.5 ± 2.4 | 5.0 ± 2.1 | 4.8 ± 2.1 | 5.4 ± 2.5* |
| Estradiol (pmol/L) | 224.2 ± 237.0 | 273.1 ± 261.9 | 228.7 ± 199.9 | 298.8 ± 308.8 |
| Aspartate Aminotransferase (U/L) | 29.5 ± 12.0 | 27.3 ± 6.4 | 28.6 ± 24.8 | 24.7 ± 6.2 |
| Alanine Aminotransferase (U/L) | 27.8 ± 18.4 | 23.2 ± 11.3 | 24.4 ± 16.0 | 22.2 ± 12.5 |
| C-Reactive protein (mg/dL) | 3.7 ± 4.1 | 3.0 ± 2.9 | 3.2 ± 3.2 | 3.0 ± 3.5 |
| Systolic Blood Pressure (mmHg) | 119.1 ± 11.8 | 120.1 ± 12.1 | 120.7 ± 11.2 | 117.6 ± 12.7 |
| Diastolic Blood Pressure (mmHg) | 71.8 ± 9.2 | 72.0 ± 8.7 | 73.6 ± 9.1 | 71.3 ± 7.9 |
| Resting Heart Rate (bpm) | 63.8 ± 7.8 | 62.8 ± 8.6 | 63.0 ± 11.0 | 62.6 ± 9.9 |

### Energy Expenditure and Intake

As shown in Table 2 a small non-significant reduction in resting metabolic rate was observed in both groups. Physical activity levels increased slightly by the end of the intervention (p<0.05), but to an equivalent extent in the two groups, without a change in subjects' activity classification. Daily energy intake was decreased in the two groups and this effect was attributable to a lower lipid consumption in the placebo group and a decrease in both lipid and carbohydrate intake in the probiotic group. Lipid intake was decreased in favor of protein in both groups. At baseline, the percentage of lipid intake during the buffet-type meal was lower in the probiotic group and this between-group difference was significantly exacerbated after the intervention despite a significant decrease in both groups.

TABLE 2
Energy metabolism (intake, expenditure and physical activity). Data are represented
as Mean ± SD, Within group significance (*= p < 0.05, **= p <
0.01), between group significance (†= p < 0.05), difference between groups at baseline (‡= p < 0.05).

| | Placebo | | Probiotic | |
|---|---|---|---|---|
| | Baseline | 3 months | Baseline | 3 months |
| | (n = 72) | (n = 52) | (n = 78) | (n = 78) |
| 24 h Energy expenditure (kcal/day) | 1692 ± 251 | 1633 ± 292 | 1676 ± 262 | 1634 ± 246 |
| Physical Activity Level | 1.46 ± 0.06 | 1.53 ± 0.13** | 1.48 ± 0.07 | 1.55 ± 0.15** |

Three-day food record

| | | | | |
|---|---|---|---|---|
| Mean Total Daily Energy intake | 2091 ± 529 | 1790 ± 383** | 2216 ± 548 | 1767 ± 343** |
| (kcal/day) | | | | |
| Mean Total Daily Carbohydrates | 245.4 ± 75.1 | 215.5 ± 50.4 | 261.5 ± 77.6 | 215.8 ± 48.4** |
| (g) | | | | |
| Mean Total Daily Lipids (g) | 76.9 ± 23.9 | 59.2 ± 18.8** | 81.9 ± 26.1 | 56.5 ± 14.8** |
| Mean Total Daily Proteins (g) | 93.3 ± 25.0 | 91.8 ± 19.6 | 93.2 ± 26.0 | 93.2 ± 23.2 |
| % Carbohydrates | 46.8 ± 7.7 | 48.2 ± 4.9 | 47.4 ± 8.0 | 48.9 ± 5.6 |
| % Lipids | 33.1 ± 5.9 | 29.5 ± 5.7* | 33.1 ± 6.5 | 28.8 ± 5.3** |
| % Proteins | 18.2 ± 4.0 | 20.8 ± 3.2** | 17.1 ± 3.4 | 21.1 ± 3.4** |

Buffet

| | | | | |
|---|---|---|---|---|
| Total Energy intake (kcal) | 922 ± 406 | 924 ± 446 | 858 ± 325 | 815 ± 291 |
| Total Carbohydrates (g) | 91.9 ± 50.5 | 93.8 ± 47.7 | 89.9 ± 40.2 | 87.2 ± 35.2 |
| Total Lipids (g) | 44.0 ± 20.8 | 43.4 ± 25.4 | 39.1 ± 17.5 | 34.9 ± 15.0 |
| Total Proteins (g) | 39.5 ± 16.7 | 39.8 ± 16.0 | 36.6 ± 12.4 | 38.0 ± 14.8 |
| % Carbohydrates | 39.5 ± 8.7 | 41.3 ± 8.2 | 41.9 ± 7.8 | 43.4 ± 10.2 |
| % Lipids | 42.8 ± 8.1 | 40.5 ± 8.8* | 40.3 ± 7.5‡ | 37.7 ± 9.1*† |
| % Proteins | 17.6 ± 4.3 | 18.2 ± 4.1 | 17.8 ± 4.4 | 18.9 ± 5.2 |

The aim of this study was to determine if a probiotic supplementation (e.g., L. rhamnosus HA-114) can improve the outcome of a weight-reducing program on body composition as well as behavioral variables related to the gut-brain axis. To this end, the probiotic supplementation in combination with dietary supervision aiming at a spontaneous decrease in energy intake was tested. As expected, the results demonstrated a significant decrease in body weight and fat in both groups, confirming the efficiency of the dietary intervention. In addition, the probiotic strain recovery from fecal samples in 95% of the participants allocated to receive the probiotic demonstrated good compliance to the present intervention.

Despite similar weight loss in both groups, the probiotic intervention improved biomarkers of metabolic health that were not observed in the placebo group. As indicated above, a significant reduction in fasting insulin levels, LDL-cholesterol and triglycerides were observed only in the probiotic group. Even if biomarker values were below a clinical diagnostic level, the favorable effects observed in this group suggest that probiotic supplementation can help prevent comorbidities related to metabolic syndrome which can ultimately lead to type II diabetes. Indeed, prevention of chronic disease has a positive impact on quality of life and life expectancy.

Leptin is a hormone produced by adipose tissue to signal to the brain the energy storage. Therefore, overweight and obesity are associated with higher circulating levels of leptin, which are expected to be lowered by a weight loss and more specifically a fat loss, as regulation of energy balance. In this study, as expected, leptin levels have been significantly decreased by the nutritional intervention and weight loss, although the addition of a probiotic treatment did not seem to have an impact on the level of decrease of leptin. Ghrelin, on the other hand, is a hormone produced by the stomach that has an opposite effect to leptin on energy balance as its basal function is to induce appetite. In this cohort, weight loss has been associated with an increase in circulating fasting ghrelin, which was only significant in the probiotic group, in a similar way to the decrease in insulin, triglycerides and LDL-cholesterol. Moreover, the decrease in daily energy intake was slightly greater in the probiotic group, which was associated with a small, non-significant accentuation of body weight loss of 0.5 kg.

### Appetite Sensation Measurements

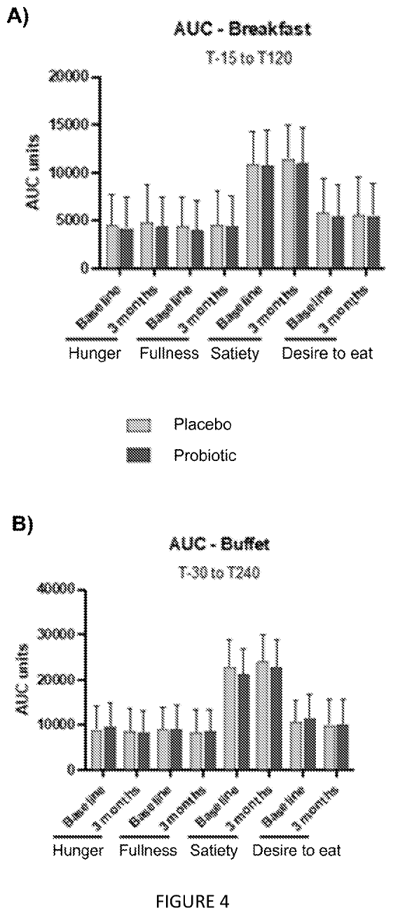

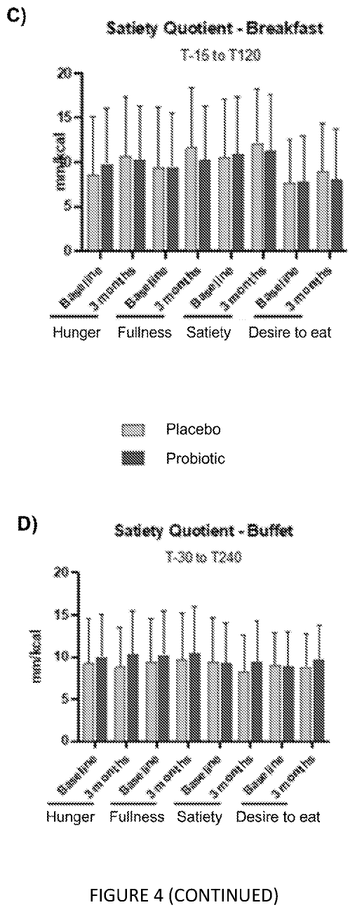

Appetite sensations were measured before and after the standardized breakfast and the buffet-type meal. Four parameters were measured with visual analog scales (VAS), namely: hunger, fullness, satiety and desire to eat. An analysis of the area under the curve (AUC) of all measurements for both breakfast and lunch revealed no significant between-group or within-group differences (FIG. 4 A-B). Similarly, no significant difference in satiety quotients between the two groups was observed for all appetite sensations (FIG. 4 C-D).

### Eating Behaviors

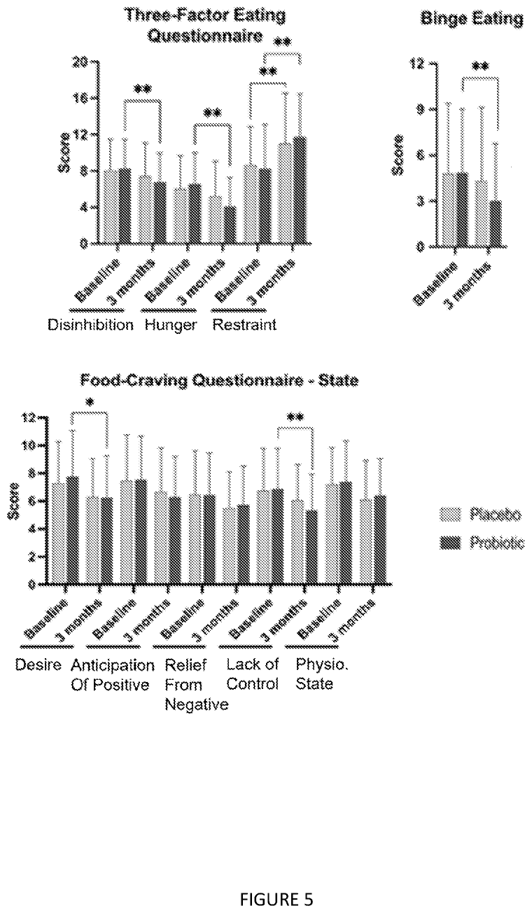

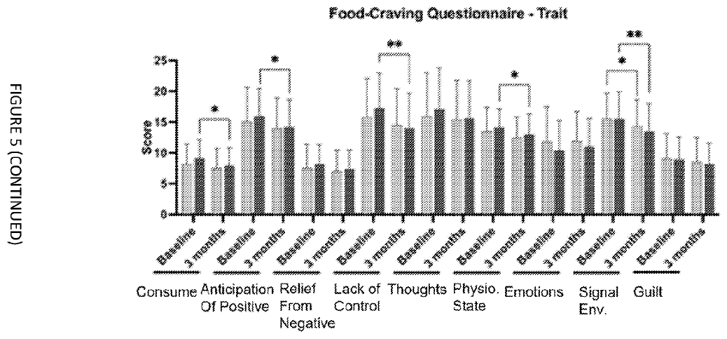

The Three-Factor Eating Questionnaire (TFEQ) revealed a decrease in disinhibition and hunger in the probiotic group only, and a between-group effect was also observed for these parameters (p=0.05 and p<0.01, respectively). Cognitive restraint increased in both groups, but this effect was more pronounced in the probiotic group, with a significant diet-treatment interaction (p=0.05). When evaluating binge eating behavior, a decrease was observed in the probiotic group only (FIG. 5 B), with an intergroup significance (p<0.01). With respect to the State-Trait Food Cravings Questionnaire (FCQ), we observed a higher number of dimensions decreasing in the probiotic group (7 out of 14) than in the placebo group (1 out of 14), suggesting a better control of food cravings in the probiotic group (FIG. 5 C-D). Interestingly, the dimension lack of control was lowered in the probiotic group both at the state and trait level with an intergroup significance (p<0.05 and p<0.01, respectively).

### Mood-Related Parameters

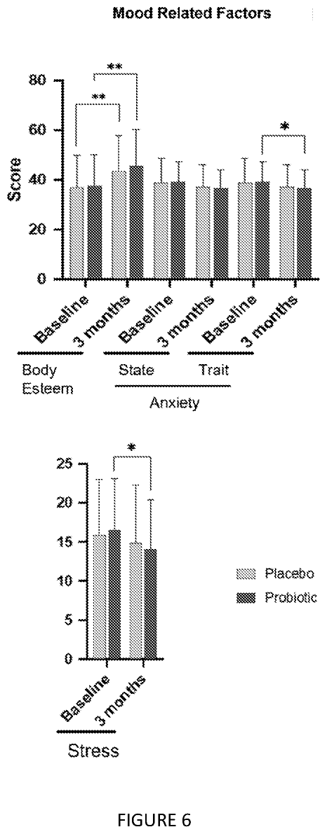

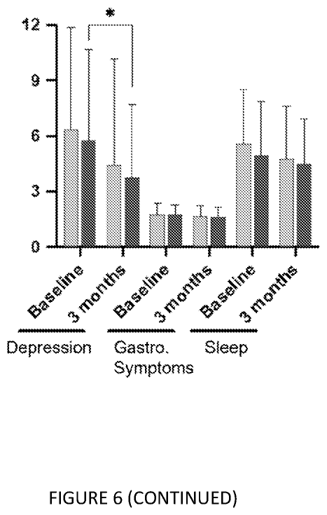

Mood-related parameters assessed throughout this study included body self-esteem, anxiety, stress and depression, as well as other factors that can impact mood, namely gastrointestinal symptoms and sleep quality (FIG. 6). An equivalent increase in body self-esteem was observed in the two groups. In addition, there was a decrease over time in anxiety (trait), perceived stress and depression in the probiotic group, although the diet-treatment interaction was not significant. Neither diet nor probiotic supplementation affected total sleep quality or overall gastrointestinal symptoms, as shown by the absence of change on these parameters before and after the intervention (FIG. 6).

There were no differences in appetite sensations at the standardized breakfast or at the buffet-type meal in either groups, despite the observed changes in circulating hormones related to hunger. The lack of change in satiety could be attributable to limitations of the visual analogue scale (VAS) or a relatively moderate weight loss which may not have been sufficient to induce clear measurable changes. Indeed, a VAS includes a question or comment which is responded using a horizontal line which describes only the extremes. Some of VAS' disadvantages include misinterpretation or diverse interpretation of the scale. Since only the extremes of the horizontal line are labelled, participants may have difficulties to know where they identify themselves within that line (Klimek et al., 2017). Hence, participants may have a similar perception but score differently increasing the variability of the results. Furthermore, the response is hard to interpret as it requires distance measurements between the participants' marks and the extremes of the line. Since two evaluators may measure the distance slightly different, there is further addition of variance. However, it is important to consider that the quantification of circulating hormones (i.e., leptin and ghrelin) and their influence on behavior and appetite control may not be representative of neurotransmitter signaling to the brain. Homeostatic and hedonic signals in the control of food intake are very complex. Indeed, a recent study has shown that moderate weight loss in individuals with obesity induces leptin and ghrelin changes while appetite sensations remained unchanged, and it was hypothesized it might be due to hormonal resistance (Hernandez Morante et al.).

It is known in the art that a weight loss due to diet restriction or surgery induces beneficial changes in eating-related behaviors. This is concordant with our observation that body self-esteem improved in both groups, suggesting that this was modulated by weight loss. Beyond this effect, we demonstrated the relevance of psychobiotics in overweight individuals subjected to a weight-reducing program. At comparable body weight loss, probiotic supplementation induced further improvements in behaviors compared to diet supervision alone. Our results, based on different validated questionnaires commonly used in the field, suggest two categories of observations supporting this notion. First, we observed a significant decrease on several parameters for which no effect was observed in the control group; these include disinhibition and hunger measured by the TFEQ, several food-cravings dimensions at the state level (desire to eat and lack of control) and trait level (intention and planning to consume food, anticipation of positive reinforcement, lack of control and physiological state), Beck depression inventory score, binge eating tendencies, perceived stress and the anxiety trait. Second, the existence of significant group treatment interactions suggests a superiority impact of the probiotic intervention on some aspects, including the increase in restraint and decrease in disinhibition, hunger, lack of control (both at the state and trait levels), lower trait of intention and planning to consume food, and binge eating. These observations suggest that participants receiving the probiotic “broke” the association of food with positive reinforcement, which facilitated the cognitive restraint to eat and reduced anxiety. In short, these participants no longer associated food with a positive outcome which facilitated the decrease in the urge to eat. Collectively, these results demonstrate a clear benefit of L. rhamnosus HA-114 supplementation on eating and mood-related behaviors associated to a healthier relationship with food and food sensations during a weight loss program.

The last parameters relating gut-brain axis measured were on wellness and quality of life. A significant lower score on the depression inventory, trait of anxiety and perception of stress was observed after the supplementation in the probiotic group, but not in the placebo group. These results point towards a beneficial effect of the probiotic on these important psychological variables. The positive modulation of the metabolic markers and the improvements on food-related behaviours, stress and depression could, if maintained, continue to contribute to a better metabolic health through better appetite control and self-esteem. Even if weight loss has been shown as an efficient way to ameliorate metabolic health, weight regain over the years is quite common and cycles of intentional weight loss and unintentional weight regain can have adverse effects, such as greater reported depressive symptoms (Quinn et al.). Therefore, by ameliorating metabolic and psychological traits, the probiotic strain could be used independently of weight loss to diminish obesity-associated comorbidities.

In conclusion, our results support the clinical relevance of probiotic supplementation during a weight loss dietary intervention in participants with overweight. Although further studies are needed to better understand the mechanisms underlying the effects of L. rhamnosus and determine conditions under which a clinical outcome may be optimized, the results of this study reveal beneficial psychological outcomes supporting the concept that probiotic supplementation could favor wellness and facilitate self-control during a diet-based weight reduction program.

### REFERENCES

Beck A T, Ward C H, Mendelson M, Mock J, Erbaugh J. An inventory for measuring depression. Arch Gen Psychiatry. 1961; 4:561-71.
Buysse D J, Reynolds C F, 3rd, Monk T H, Berman S R, Kupfer D J. The Pittsburgh Sleep Quality Index: a new instrument for psychiatric practice and research. Psychiatry Res. 1989; 28(2):193-213.
Cohen S, Kamarck T, Mermelstein R. A global measure of perceived stress. J Health Soc Behav. 1983; 24(4):385-96.
Drapeau V, Blundell J, Therrien F, Lawton C, Richard D, Tremblay A. Appetite sensations as a marker of overall intake. Br J Nutr. 2005; 93(2):273-80.
Drapeau V, King N, Hetherington M, Doucet E, Blundell J, Tremblay A. Appetite sensations and satiety quotient: predictors of energy intake and weight loss. Appetite. 2007; 48(2):159-66.
Dohle S, Diel K, Hofmann W. Executive functions and the self-regulation of eating behavior: A review. Appetite. 2018 May 1; 124:4-9.
Ford A L, Nagulesapillai V, Piano A, Auger J, Girard S A, Christman M, Tompkins T A, Dahl W J. Microbiota stability and gastrointestinal tolerance in response to a high-protein diet with and without a prebiotic, probiotic, and synbiotic: A randomized, double-blind, placebo-controlled trial in older women. Journal of the Academy of Nutrition and Dietetics. 2020 Apr. 1; 120(4):500-16.
Green S M, Delargy H J, Joanes D, Blundell J E. A satiety quotient: a formulation to assess the satiating effect of food. Appetite. 1997; 29(3):291-304.
Grompone, G., et al., Anti-inflammatory Lactobacillus rhamnosus CNCM I-3690 strain protects against oxidative stress and increases lifespan in Caenorhabditis elegans. PLoS One, 2012. 7(12): p. e52493.
Frayn K N. Calculation of substrate oxidation rates in vivo from gaseous exchange. J Appl Physiol Respir Environ Exerc Physiol. 1983; 55(2):628-34.
Hawkins R C, 2nd, Clement P F. Development and construct validation of a self-report measure of binge eating tendencies. Addict Behav. 1980; 5(3):219-26.
Hernandez Morante J J, Diaz Soler I, Munoz J S G, Sanchez H P, Barbera Ortega M D C, Martinez C M, et al. Moderate Weight Loss Modifies Leptin and Ghrelin Synthesis Rhythms but Not the Subjective Sensations of Appetite in Obesity Patients. Nutrients. 2020;12(4)
Hill A J, Blundell J E. Macronutrients and satiety: the effects of a high-protein or high-carbohydrate meal on subjective motivation to eat and food preferences. Nutrition and behavior (USA), 1986; 3(2):133-134
Klimek, L., Bergmann, K. C., Biedermann, T., Bousquet, J., Hellings, P., Jung, K., Merk, H., Olze, H., Schlenter, W., Stock, P. and Ring, J., 2017. Visual analogue scales (VAS): Measuring instruments for the documentation of symptoms and therapy monitoring in cases of allergic rhinitis in everyday health care. Allergo journal international, 26(1), pp.16-24.
Labarre A., Guitard E., Tossing G. et al. Probiotic Lacticaseibacillus rhamnosus HA-114 suppresses age-dependent neurodegeneration via mitochondrial beta-oxidation, 24 Sep. 2020, PREPRINT (Version 1) available at Research Square [https://doi.org/10.21203/rs.3.rs-59192/v1]
Luppino F S, de Wit L M, Bouvy P F, Stijnen T, Cuijpers P, Penninx B W, et al. Overweight, obesity, and depression: a systematic review and meta-analysis of longitudinal studies. Arch Gen Psychiatry. 2010; 67(3):220-9.
Markowiak P, Śliżewska K. Effects of Probiotics, Prebiotics, and Synbiotics on Human Health. Nutrients. 2017;9(9):1021.
Mendelson B K, Mendelson M J, White D R. Body-esteem scale for adolescents and adults. J Pers Assess. 2001; 76(1):90-106.
Nijs I M, Franken I H, Muris P. The modified Trait and State Food-Cravings Questionnaires: development and validation of a general index of food craving. Appetite. 2007; 49(1):38-46.
Quinn D M, Puhl R M, Reinka M A. Trying again (and again): Weight cycling and depressive symptoms in U.S. adults. PLoS One. 2020 Sep. 11; 15(9):e0239004.
Sanchez M., et al., Effects of a Diet-Based Weight-Reducing Program with Probiotic Supplementation on Satiety Efficiency, Eating Behaviour Traits, and Psychosocial Behaviours in Obese Individuals. Nutrients. 2017 Mar. 15; 9(3):284.
Sarkar A., Lehto S. M., Harty S., Dinan T. G., Cryan J. F., Burnet P. W. Psychobiotics and the manipulation of bacteria-gut-brain signals. Trends in neurosciences. 2016 Nov. 1; 39(11):763-81.
Soczynska J K, Kennedy S H, Woldeyohannes H O, Liauw S S, Alsuwaidan M, Yim C Y, McIntyre R S. Mood disorders and obesity: understanding inflammation as a pathophysiological nexus. Neuromolecular medicine. 2011 Jun. 1; 13(2):93-116.
Spielberger C, Goruch R, Lushene R, Vagg P, Jacobs G. Manual for the state-trait inventory STAI (form Y). Mind Garden, Palo Alto, CA, USA. 1983.
Stunkard A J, Messick S. The three-factor eating questionnaire to measure dietary restraint, disinhibition and hunger. J Psychosom Res. 1985; 29(1):71-83.
Svedlund J, Sjödin I, Dotevall G. GSRS—a clinical rating scale for gastrointestinal symptoms in patients with irritable bowel syndrome and peptic ulcer disease. Dig Dis Sci. 1988; 33(2):129-34.

1.-13. (canceled)

14. A method of favoring wellness and facilitating self-control in a subject in need thereof who had been on a restrictive diet, is currently on the restrictive diet and/or intends to be on the restrictive diet, the method comprises administering to the subject a probiotic composition comprising an effective amount of a probiotic product and an acceptable carrier, wherein the probiotic product comprises at least one of a population of probiotic bacteria from a Lactobacillus genus and/or a microbial component derived from the population of the probiotic bacteria.

15. The method of claim 14 for preventing, limiting and/or suppressing at least one negative psychological effect associated with the restrictive diet.

16. The method of claim 15, wherein the at least one negative psychological effect comprises disinhibition, hunger, a desire to eat, lack of control, intention and planning to consume food, an anticipation of positive reinforcement with food consumption, a binge eating tendency, perceived stress and the anxiety trait, or a combination thereof.

17. The method of claim 14, wherein the subject has been determined to need and/or wishes to seek psychological support to complete the restrictive diet.

18.-33. (canceled)

34. The method of claim 14, wherein the subject is an overweight subject, an obese subject or the subject has a healthy weight.

35. The method of claim 14, wherein the subject is a human subject.

36. The method of claim 14, wherein the subject is currently on the restrictive diet.

37. The method of claim 14, comprising administering the probiotic composition during the restrictive diet.

38. The method of claim 14, wherein the population of probiotic bacteria comprises Lactobacillus rhamnosus (L. rhamnosus).

39. The method of claim 37, wherein the population of probiotic bacteria comprises at least one of L. rhamnosus R0011 (LHS), L. rhamnosus HA-114 (LHS), L. rhamnosus HA-500 (LHS), L. rhamnosus R0049 (LHS), L. rhamnosus R0343 (LHS), or L. rhamnosus R1039 (LHS).

40. The method of claim 38, wherein the population of probiotic bacteria comprises L. rhamnosus HA-114 (LHS).

41. The method of claim 14, wherein the population of the probiotic bacteria is provided at a daily dosage of from about 1×10⁵ to about 1×10¹² colony-forming units (cfu) total bacteria.

42. The method of claim 14, wherein the probiotic composition is provided in the form of a freeze-dried power, a tablet, a capsule, a pill, a suspension, an emulsion, a liquid preparation, a gel, a syrup, a cream or an inhalable formulation.

43. The method of claim 14, wherein the probiotic composition is provided as a food product or a drink product.

44. A method of formulating a probiotic composition for favoring wellness and facilitating self-control in a subject in need thereof who had been on a restrictive diet, is currently on the restrictive diet and/or intends to be on the restrictive diet, wherein the method comprises combining (i) at least one of a population of probiotic bacteria from a Lactobacillus genus and/or a microbial component derived from the population of the probiotic bacteria with (ii) an acceptable carrier to prepare the probiotic composition.
